(19) United States
(12) Patent Application Publication
(10) Pub. No.: US 2009/0192437 A1
(43) Pub. Date: Jul. 30, 2009

(54) WEARABLE PHOTOACTIVATOR FOR OCULAR THERAPEUTIC APPLICATIONS AND USES THEREOF

(75) Inventors: Robert Soltz, Spring Valley, NY (US); Barbara Ann Soltz, Spring Valley, NY (US); Ashley Behrens, Sparks Glencoe, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/236,986

(22) Filed: Sep. 24, 2008

Related U.S. Application Data
(60) Provisional application No. 60/994,979, filed on Sep. 24, 2007.

(51) Int. Cl. A61F 9/00; A61M 37/00

(52) U.S. Cl. 604/20; 607/88

(57) ABSTRACT

The invention provides a wearable device for delivery of light of a desired wavelength and power to the cornea of a subject. The device includes a frame for attachment of a light source housing which includes a light source and a lens positioned in the housing to allow light to be directed to the eye of the subject, and the light source is operably linked to a power source. The invention provides method for the prevention and treatment of ocular disease including infection, neoplasia, and corneal dystrophies. The device of the invention can be used in conjunction with photoactive therapeutic agents.

### RELATED APPLICATIONS

This application claims priority to U.S. Provisional Patent Application Ser. No. 60/994,979 filed on Sep. 24, 2007, which is incorporated by reference herein in its entirety.

### STATEMENT OF RIGHTS TO INVENTIONS MADE UNDER FEDERALLY SPONSORED RESEARCH

This work was supported in part by the National Eye Institute grant numbers 1R43 EY 015055. The government has certain rights in the invention.

### BACKGROUND OF THE INVENTION

Infectious keratitis is probably one of the most feared diseases of the cornea. Depending on the pathogen responsible of the infection, the prognosis for visual rehabilitation may be poor. Topical antibiotics and anti-infectives are prone to resistance development, since microorganisms have mechanisms to transform and avoid the effects of these medications. Systemic administration of anti-infective agents is virtually useless, as therapeutic levels are not reached in the area where the infection develops. Therefore, the cornea constitutes an ideal tissue to harbor living microorganisms, as it is bathed by the tear film with nutrients, and lacks of vessels to allow the protective systems of the body react against pathogenic microorganisms.

Recently, several outbreaks of infectious keratitis have been reported in the U.S. and worldwide (CDC Health Advisory. Early Report of Serious Eye Infections Associated with Soft Contact Lens Solution. (http://www2a.cdc.gov/HAN/ArchiveSys/ViewMsgV.asp?AlertNum=00260) and Singapore Ministry of Health. Increasing incidence of contact lens related fungal corneal infections (update 3). Feb. 21, 2006 http://www.moh.gov.sg/corp/about/newsroom/pressreleases/details.do?id=36077601). Paradoxically, some of them have been related to the use of contact lens disinfecting solutions, and have been particularly resilient to traditional treatment.

### SUMMARY OF THE INVENTION

The invention provides a wearable ocular photoactivator device. The device to be worn by a subject includes a frame and a light source that is directed towards the eye of the subject when the subject is wearing the device. The light source is contained within a housing is connected to the frame. The housing includes the light source contained within the housing, and one or more lenses that can be aligned with emitted light from the light source. The lenses focus the light onto the cornea of the eye, providing a spot of light of a defined, predetermined size at a particular distance from the eye. The spot of light can be directed to a specific portion of the eye, e.g., the cornea, while avoiding other parts of the eye, preventing, or limiting the damage to the eye. The device also includes a power source operably connected to the light source to provide power to the device. In an embodiment, the device includes a structure for attachment of the frame a head of the subject, such as arms or bands or both.

The invention provides an adjustable mount for the light assembly housing. In various embodiments of the invention, the housing can be adjusted in one, two, or three dimensions relative to the eye of the subject to be treated. In certain embodiments, the housing can be rotated within the mount. The device can include a measuring device or distance gauge to facilitate the adjustment of the device on the face of the subject.

The invention provides the use of any light source that can provide the appropriate wavelength and power to practice the methods of the invention. For example, lights for use with the device of the invention include, but are not limited to light emitting diodes (LED), laser diodes, frequency tripled Nd:Yag solid state lasers, dye lasers, quartz lamps, fluorescent lamps, Nernst glowers, Tungsten-Halogen lamps, and discharge lamps. The wavelength of emitted light for use in the invention includes ultraviolet, visible, infrared, and x-ray. In a preferred embodiment, the wavelength of light emitted is UV-A (380 nm -315 nm).

The invention provides a light source housing having one or more lenses. In some embodiments, the housing includes lenses of more than one size. Such lens housings can be rotated to allow for the alignment of the desired lens(es) with the light source(s). In alternative embodiments, the lens housing attached to the proximal end of the housing can be easily exchanged to provide lenses of the desired size. In an embodiment, the angle of the lenses can be adjusted in the lens housing. The housing can also include an opening that can serve one or more purposes. The opening can be used by an ophthalmologist to align the housing with the appropriate portion of the eye of the subject. The ophthalmologist can introduce the photoactive therapeutic agent through the opening in the housing, using an automated dropper device, or manually using, for example, a medicine dropper. The opening can also be used by the subject to allow the subject to watch television or other form of visual entertainment during the treatment. The eye of the subject not being treated can be covered with an occluder that optionally includes an opening for use by the ophthalmologist, the subject, or both.

The invention further provides a wearable ocular photoactivator device having a NIR source and a phototransistor to detect light emitted by the light source. This allows for the device to detect if the lens(es) and light source(s) are in proper alignment, and/or to detect the size lens aligned with the light source.

The invention further provides the use of the devices of the invention for the treatment of ocular disorders or diseases, particularly diseases and disorders of the cornea such as infections, corneal dystrophies, and corneal neoplasia. The methods include exposing the eye of a subject to light using the device of the invention by positioning the device on the face of the subject to direct light onto the eye, preferably onto the cornea of the subject, and providing power to a device of the invention such that light of the desired wavelength and power is provided to the eye, preferably the cornea for the desired amount of time. In a preferred embodiment, the eye of the subject is contacted with at least one photoactive therapeutic agent prior to exposure to light of the desired power and wavelength. Preferably, the photoactive agent is delivered topically to the surface of the eye. In certain embodiments, the invention includes repeated administration of the phototherapeutic agent with multiple rounds of light administration between the repeated administrations of the agent. In an embodiment, the subject is selected for having an ocular disease, particularly a corneal disease. In an embodiment, the subject is monitored for amelioration of at least one sign or symptom of the disease or disorder.

The invention provides for the use of essentially any photoactive therapeutic agent with the desired activity, e.g., cell killing activity, including anti-pathogen activity and anti-neoplastic activity, and/or cross-linking activity to rigidify the cornea. In a preferred embodiment, the activity of the photoactive therapeutic agent is a result of the activity of the agent as a crosslinking agent, or the activity of the agent as a generator of singlet oxygen species. Agents for use in the methods of the invention include, but are not limited to, riboflavin, psoralen, lumiflavin, lumichrome, rose Bengal, eosin, courmarin, sparfloxacin, fluorescein, ficusin, psoberan, Toluidine Blue O, methylene blue, and thionin.

The invention further provides kits for practicing the methods of the invention. Kits of the invention include, for example, a device of the invention and a photoactive therapeutic agent. Kits can further include instructions for use and appropriate packaging.

### DEFINITIONS

By “ameliorate” is meant decrease, suppress, attenuate, diminish, arrest, or stabilize the development or progression of a disease. Amelioration can require administration of more than one dose.

By “diagnosing” as used herein refers to a clinical or other assessment of the having a disease, disorder, or condition based on the presence of at least one sign or symptom of the disease, disorder, or condition. Typically, diagnosing using the method of the invention includes the observation of the subject for other signs or symptoms of the disease, disorder, or condition in addition to detection of a loss-of-function mutation in a gene that makes the subject susceptible to a particular disease or condition.

“Distal” is meant the end or portion of the device furthest away from the subject, particularly the eye of the subject, when worn. It is understood that distal can be a relative term that one portion of the device is further away, i.e., more distal, from the subject than another portion of the device.

By “effective dose” is meant a sufficient amount of light exposure, with or without administration of a sufficient amount of one or more photoactive therapeutic agent(s), to result in a decrease of the incidence of a disease or disorder, or to result in a decrease of at least one sign or symptom of a disease or disorder. The effective dose of each light and photoactive therapeutic agent in the methods of the invention can readily be determined by one of skill in the art, such as an ophthalmologist. An effective dose is a sufficient dose to ameliorate at least one sign or symptom of a disease or condition to be treated using a device or method of the invention.

As used herein, “fluence” or “integrated flux” is defined as the number of particles that intersect a unit area . Its units are m⁻² (number of particles per meter squared). In particular, it is used to describe the strength of a radiation field, in which case the unit used is J/m². It is considered one of the fundamental units in dosimetry. In laser medicine, the fluence usually refers to the “Power Density” or “Energy Density” of a laser at the emitter tip. The higher the fluence, the more “cutting power” a laser has.

As used herein, “infection” is understood as the state produced by the establishment of an infective or pathogenic organism in or on a suitable host or subject; or a disease resulting from infection.

As used herein, “infectious keratitis” is understood to be any infection of the cornea, i.e., the front part of the eye, including, but not limited to, amoebic keratitis, usually caused by Acanthamoeba; bacterial keratitis, usually caused by Staphylococcus aureus or Pseudomonas aeruginosa; fungal keratitis, for example caused by Fusarium; and viral keratitis which can be caused by a Herpes virus such as Herpes simplex or Herpes zoster.

As used herein, “keratoconus” is understood as a degenerative non-inflammatory disorder of the eye in which structural changes within the cornea cause it to thin and change to a more conical shape than its normal gradual curve. Keratoconus can cause substantial distortion of vision, with multiple images, streaking and sensitivity to light all often reported by the patient. Keratoconus is the most common dystrophy of the cornea. Other corneal dystrophies include, but are not limited to the anterior part of the cornea (map-dot-fingerprint dystrophy, Meesman dystrophy, Reis-Bucklers dystrophy, Thiel-Behnke dystrophy), the intermediate part of the cornea (macular dystrophy, granulardystrophy, lattice dystrophy, Schnyder dystrophy) and the posterior part of the cornea (posterior polymorphous dystrophy, farinata dystrophy, Fuchs dystrophy).

As used herein, “light source” and the like are understood as a device that provides light energy in a visible (400-750 nm) or invisible (e.g., UV-A, UVB, infrared, x-ray) range. In certain embodiments, the power, and wavelength of the light energy provided can be modulated or operate in a continuous mode. In certain embodiments, the wavelength or range of wavelengths of light provided is fixed for a specific light source. In certain embodiments, the power of the light source is fixed. In certain embodiments, the light source is a light emitting diode (LED), laser diode, frequency tripled Nd:Yag solid state lasers, dye lasers, quartz lamps, fluorescent lamps, Nernst glowers (ceramic) and Tungsten-Halogen lamps, discharge lamps (e.g. carbon arcs, high pressure Xenon arc lamps or Krypton arc lamps). Any light source that provides an appropriate wavelength, or range of wavelength, and an appropriate power can be used in the device and methods of the invention.

As used herein, “obtaining” means purchasing, making, or otherwise coming into possession of.

By “ocular” is meant of or relating to the eye.

By “pathogen” is meant an organism (e.g., bacteria, virus, mycoplasm, parasite, yeast, fungus, aeomeba) that is not normally present in or on a subject, particularly a human subject, and the presence of the organism is detrimental or potentially detrimental to the subject.

As used herein, “photoactive therapeutic agent” is understood as a compound that upon exposure to an appropriate wavelength of light is “activated” and gains a new function not present in the molecule, or present at a much lower level in the molecule, prior to light exposure. For example, riboflavins, eosin, courmarin, sparfloxacin, fluorescein, lumichrome, lumiflavin, ficusin, psoberan, or tricyclic furocoumarins such as psoralen, etc. that absorb light in the UV-A range, activating anti-pathogenic activity in the compounds, are referred to herein as UV-A photoactive therapeutic agents. For example, exposure of a photoactive therapeutic agent to an appropriate wavelength of light can result in the release of free radicals that can have anti-pathogenic activity, or promote cross-linking of proteins and/or nucleic acids providing anti-pathogenic activity. Photoactive agents can intercalate bonds in collagen fibers producing cross-links upon activation of the compounds with light, stiffening the cornea. Photoactive agents that can be excited by light in the visible spectrum include rose Bengal (PolySciences), Toluidine Blue O (Sigma-Aldrich, molecular formula: C₁₅H₁₆ClN₃S), methylene blue, and fluorescent polyimides such as thionin (Sigma-Aldrich) producing singlet oxygen (active oxygen species). Such photoactive therapeutic agents can also produce free radicals that cause damage to the pathogens. As used herein, a photoactive therapeutic agent is a pharmaceutically acceptable compound that is safe for administration to a mammal, preferably a human, for the route of administration (e.g., topical, ocular administration). Photoactive therapeutic agents for use with the method of the invention are well known and include, for example, the light activated antimicrobial and antiviral agents provided in U.S. Pat. No. 6,239,048 (incorporated herein by reference). The therapeutic agent is in a pharmaceutically acceptable carrier for ocular administration (e.g., normal saline, water, buffered phosphate solution, dextran 500, hypertonic saline, hypotonic saline, hyaluronic acid, polyvinyl acid, glycerine, methylcellulose, etc.). Methods to test and confirm antimicrobial activity in response to activation by light is well within the ability of those of skill in the art.

Examples of wavelengths for excitation of various photoactive therapeutic agents are in the table below:

| Chromophore | Max Absorption λ (nm) |
|---|---|
| eosin | 518 |
| rose bengal | 559 (ethanol) |
| fluorescein | 500 |
| courmarin | 373 |
| psoralen | UVA (mercury/xenon source) |
| riboflavin | 365, 450 |
| lumichrome | 365, 436 |
| lumiflavin | 365, 445 |

It is understood that the wavelength of light absorption and emission is dependent, for example, on pH and other conditions. Such considerations are well understood by those of skill in the art.

“Providing,” refers to obtaining, by for example, buying or making the, e.g., device or photoactive therapeutic agent. The material provided may be made by any known or later developed biochemical or other technique.

By “proximal” is meant the end or portion of the device closest to the subject, particularly the eye of the subject, when worn. It is understood that proximal can be a relative term that one portion of the device is closer, i.e., more proximal, to the subject than another portion of the device.

The term “subject” includes organisms which are capable of suffering from a disease of interest that could otherwise benefit from a treatment using the devices and/or methods of the instant invention. The term “non-human animals” of the invention includes all vertebrates, e.g., mammals, e.g., sheep, dog, cow, and primates including non-human primates; e.g., rodents, e.g., mice, , and non-mammals, e.g., chickens, amphibians, reptiles, etc. A human subject can be referred to as a patient.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

By “selecting” for example “selecting a subject in need of treatment” is meant identifying an individual for treatment using devices and/or methods of the instant invention by the presence of one or more signs or symptoms of a disease or disorder amenable to treatment with the devices or methods of the instant invention. Selecting can also include identification of an appropriate photosensitive therapeutic agent, light exposure time, wavelength of light, light exposure power, etc for use with the devices and methods of the instant invention.

The term “treated,” “treating” or “treatment” includes the diminishment or alleviation of at least one symptom associated or caused by the state, disorder, condition, or disease being treated. For example, treatment can be diminishment of one or several symptoms of a disorder or complete eradication of a disorder. Treatment can also include prophylaxis (i.e., prevention). Treatment can result in amelioration of a disease. Treatment and prophylaxis can require administration of more than one dose.

As used herein, “ultraviolet light” or “UV light” is light energy that is within the range of wavelengths in the ultraviolet range. Wavelengths for various types of UV light are presented in the table below.

| | | Wavelength range | Energy |
|---|---|---|---|
| Name | Abbreviation | in nanometers | per photon |
| Ultraviolet A, long | UV-A | 380 nm-315 nm | 3.10-3.94 eV |
| wave, or black light | | | |
| Near | NUV | 400 nm-300 nm | 3.10-4.13 eV |
| Ultraviolet B or | UV-B | 315 nm-280 nm | 3.94-4.43 eV |
| medium wave | | | |
| Middle | MUV | 300 nm-200 nm | 4.13-6.20 eV |
| Ultraviolet C, short | UV-C | 280 nm-100 nm | 4.43-12.4 eV |
| wave, or germicidal | | | |
| Far | FUV | 200 nm-122 nm | 6.20-10.2 eV |
| Vacuum | VUV | 200 nm-10 nm | 6.20-124 eV |
| Extreme | EUV | 121 nm-10 nm | 10.2-124 eV |

It is understood that the wavelength of light is selected based on the wavelength or wavelengths of light absorbed by the photoactive therapeutic agent.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive.

Unless specifically stated or obvious from context, as used herein, the terms “a”, “an”, and “the” are understood to be singular or plural.

As used herein, “about” is understood to mean approximately or reasonably close to, and within the tolerances generally accepted in the specific experiment or result, for example within two standard deviations of the mean of a specific result. For example, about can be understood as a variation of 10% or less, 7% or less, 5% or less, 2% or less, or 1% or less.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Each patent, patent application, or reference cited herein is hereby incorporated by reference as if each were incorporated by reference individually.

### BRIEF DESCRIPTION OF THE DRAWINGS

FIG. 1 shows a view of an embodiment of the device of the invention;

FIG. 2 shows an alternate view of the embodiment of the invention shown in FIG. 1;

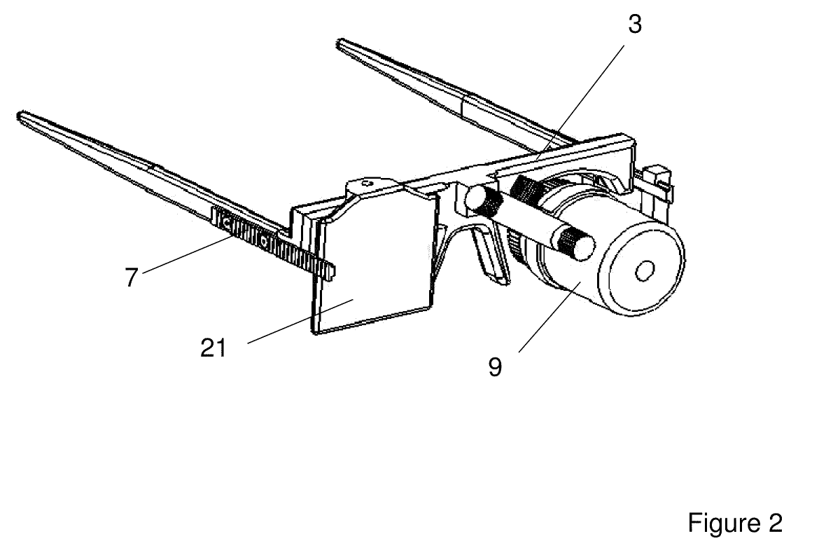

FIG. 3 shows a view of an embodiment of a device of the invention with an opening in the occluder;

FIG. 4 shows a view of an embodiment of the proximal end of the light source housing with two different size lenses and an opening through the length of the housing;

FIG. 5 shows a cross sectional view of the light source housing with a fluid delivery device inserted through the opening in the housing through the length of the housing, and attached to a box for dripper control;

FIG. 6 shows an embodiment of the device of the invention from the proximal (i.e. patient) perspective with a light source housing containing multiple lenses;

FIG. 7 shows an embodiment of the light source housing;

FIG. 8 shows an embodiment of the interior of the light source housing and is the light spot size detector assembly;

FIG. 9 shows a transparent view of the light source housing assembly;

FIG. 10 shows a cross section of the light source housing cut along the length of the housing through the center of the housing, and shows the light path for the light beam convergence (8 mm diameter);

FIG. 11 shows a cross section of the light source housing, cut along the length of the housing through the center of the housing assembly showing the light path convergence of the 5 mm diameter beam;

FIGS. 12A and 12B show the power selection mechanism in the light source housing in both the open transmission (A) and closed transmission (B) position;

FIGS. 13A and 13B show control circuit schematics of the power selection mechanism in FIGS. 12A and 12B with an open slot providing a low signal to the control box (A) and a closed slot providing a high signal to the control box (B);

FIGS. 14A and 14B show control circuit schematics in the control box of the power selection mechanism in FIGS. 12A and 12B with an open slot providing a low signal to the control box (A) and a closed slot providing a high signal to the control box (B) using a CMOS switch or relay; and

FIG. 15 shows an embodiment of a light source control box.

### DETAILED DESCRIPTION OF THE INVENTION

The invention provides devices and methods for treatment of diseases and disorders of the eye, particularly the cornea. The device to be worn by a subject includes a frame to which a light source contained in a housing is connected. The light source can be adjusted for delivery of a specific wavelength or range of wavelengths of light to the cornea of the same subject, to either the surface of the cornea, or an internal layer of the eye. The light is delivered to the eye at a specific power over a specific area, typically a round area.

The invention provides a device and therapeutic method using a combination of light and a photoactive molecule for the treatment of infectious keratitis. The invention includes the use of a photoactive therapeutic agent such as riboflavin (Vitamin B-2), combined with the UV-A exposure directly to the cornea. This treatment is similar to an approach to induce corneal stiffening by means of chemically binding collagen bands within the cornea in a process called cross-linking. Using this method, ophthalmologists have been able to stop the progression of keratoconus and other corneal ectasias, inducing more rigidity to the cornea to avoid the corneal deformation on these conditions (Wollensak G. Crosslinking treatment of progressive keratoconus: new hope. Curr Opin Opthalmol. 2006; 17:356-60, incorporated herein by reference). On the other hand, using a similar approach, researchers have been able to sterilize biological fluids using the oxidative byproducts of photoactivated riboflavin after UV-A light exposure. Riboflavin degrades to free-radicals, which affect DNA and RNA present in cells and viral particles (Ruane P H, Edrich R, Gampp D, Keil S D, Leonard R L, Goodrich R P. Photochemical inactivation of selected viruses and bacteria in platelet concentrates using riboflavin and light. Transfusion. 2004; 44:877-85, incorporated herein by reference). In one study, this method was effective against a parasite (Leishmania donovani) that infect humans (Cardo L J, Rentas F J, Ketchum L, Salata J, Harman R, Melvin W, Weina P J, Mendez J, Reddy H, Goodrich R. Pathogen inactivation of Leishmania donovani infantum in plasma and platelet concentrates using riboflavin and ultraviolet light. Vox Sang. 2006; 90:85-91, incorporated herein by reference). In this invention riboflavin and UV-A combination is a treatment method for a variety of infections of the cornea. Due to the universal nature of the target in this technology affecting DNA and RNA, the device and method can be used for the treatment of essentially any corneal infection as all infectious agents include nucleic acids. The device and method can also be used for neoplastic diseases of the cornea by focusing the light at the appropriate portion of the cornea, damage to adjacent tissues can be mitigated.

Light can be administered alone for therapeutic purposes. Alternatively, the light can be used in conjunction with a photoactive therapeutic agent which can be activated by light of different wavelengths, for example UV-A in the case of UV-A photoactive therapeutic agents such as riboflavin, or psoralen or other tricyclic furocoumarins. Such agents can be used as broad spectrum anti-pathogen agents as their action is dependent on intercalation in nucleic acid of the organisms, and/or by the generation of free radicals that destroy the pathogens by causing structural damage to macromolecules in the cells. Although the method can result in damage of the corneal epithelium, the high rate of turnover of the cells prevents any significant or long term damage of the eye.

It is understood that the photoactive agents for use in the methods of the invention can be unstable upon exposure to light. The invention includes readministration of the photoactive agent at one or more times during a treatment. The frequency of readministration required can depend on the energy of light administered and the area over which the light is administered. The invention also includes the use of components and methods to protect the photoactive agents from light, e.g., opaque tubing, agent reservoirs, drippers, etc. Such considerations are well understood by those of skill in the art.

UV-A photoactive agents can also be used for the treatment of corneal dystrophies, especially those which may have a component related to deposition of material produced by keratocytes, since the treatment can be adjusted in such a way that a controlled removal of keratocytes from the corneal stroma can be achieved. The depth of the effect can be adjusted by changes in the concentration of the photochemical and/or the fluence of the light. The effective dose of the photoactive agent and/or the light will need to be greater (e.g., higher concentration of photoactive agent, longer or higher power light exposure) for the treatment of corneal dystrophies as compared to infections. The dose of agent and light is limited by the posterior layer of the cornea (endothelium), which needs to be protected from the toxic radicals. Damage to endothelium results in serious damage to the cornea. It is possible that higher concentrations of agent may actually be protective of the eye by absorbing more of the light, particularly UV light, when used with the device and methods of the invention. Such considerations and variations in the use of the device would be well understood by those of skill in the art.

Moreover, due to the universal nature of the target in this technology affecting DNA and RNA, this treatment may also be used for neoplastic diseases by focalized exposure of the treatment to the tumor area. There is no selectivity, the treatment may also destroy healthy cells, but in this case the focal exposure will warrant the destruction of the targeted cells. In treatment of neoplasia, the damage of tissue adjacent to treatment areas can be an acceptable mode of treatment (e.g., radiation therapy).

The cornea is a is the transparent front part of the eye that covers the iris, pupil, and anterior chamber. Together with the lens, the cornea refracts light, and as a result helps the eye to focus, accounting for approximately 80% of the eye's optical power. The clarity and rigidity of the cornea are essential for its function.

The human cornea, like that of other primates, has five layers. The structural rigidity, shape, and clarity of the cornea are required for site and visual acuity.

The outer layer of the cornea is the corneal epithelium. It is a thin epithelial multicellular tissue layer (stratified squamous epithelium) of fast-growing and easily-regenerated cells, kept moist with tears. Irregularity or edema of the corneal epithelium disrupts the smoothness of the air-tear film interface, the most significant component of the total refractive power of the eye, thereby reducing visual acuity. It is continuous with the conjunctival epithelium is composed of about 6 layers of cells which are shed constantly on the exposed layer and are regenerated in the basal layer.

Adjacent to the corneal epithelium is Bowman's layer, a tough layer that protects the corneal stroma, consisting of irregularly-arranged collagen fibers, essentially a type of stroma. It is eight to 14 microns thick.

Corneal stroma (or substantia propria) is a thick, transparent middle layer, consisting of regularly-arranged collagen fibers along with sparsely populated keratocytes. The corneal stroma consists of approximately 200 layers of type I collagen fibrils. Ninety percent of the corneal thickness is composed of stroma.

Descemet's membrane is a thin acellular layer that serves as the modified basement membrane of the corneal endothelium.

Corneal endothelium is a simple squamous or low cuboidal monolayer of mitochondria-rich cells responsible for regulating fluid and solute transport between the aqueous and corneal stromal compartments.

The wearable photoactivator device of the invention allows for the delivery of light to the cornea particularly for prevention or treatment of a disease or condition of the eye, particularly the cornea. The device allows for the positioning of a light source over the eye of a subject. The device can be secured to the subject's face and adjusted to provide light at the appropriate spot or spots on the cornea. The light is focused to a desired spot on the cornea. The therapeutic agent preferentially absorbs this light, preventing any light from penetrating to or being transmitted to other areas of the eye. In addition, in the methods of the invention, the therapeutic agent will release reactive oxygen species including singlet oxygen which is one acknowledged mechanism for destroying microbes and simultaneously forming new molecular bi-products which tend to mask the light from penetrating to internal structures of the eye, thereby protecting the eye. However, the invention can include the use of masks or other barriers to protect portions of the eye depending on the portion of the cornea that needs to be treated and its proximity to other parts of the eye (e.g., iris).

It is understood that the device can be made in various sizes for use on subjects of different sizes, e.g., human adults and children, or animals. The specific working distance of the housing to the eye is a matter of choice by the user within a range, for example, within about 1-5 cm, about 1.5-4 cm, about 2-3 cm from the eye. The specific working distance can be adjusted for the comfort of the subject. It is understood that the device of the invention can be used for applications other than those specifically provided herein wherein the application includes the exposure of the eye to specific wavelength or range of wavelengths of light, particularly exposure of the cornea to a specific wavelength or range of wavelengths of light for a period of time, particularly a period of time longer than is convenient to have a subject remain sufficiently still, e.g. more than about 10 seconds, more than about 20 seconds, more than about 30 seconds, more than about 1 minute, more than about 2 minutes.

The device is easy to use by a user, such ophthalmologists, is relatively simple and compact, and may have reduced cost as compared to other light delivery devices. The apparatus may also facilitate a reduced patient recovery time. The device may also be more comfortable and convenient for both the patient and the ophthalmologist by allowing the patient to move his/her head during the period of light exposure of the cornea, which may be extensive (from about 1 minute, 2 minutes, 5 minutes to about at least 5 minutes, 10 minutes, 15 minutes, 20 minutes, 30 minutes, 45 minutes, 60 minutes, or 90 minutes, or more).

The light may be delivered continuously or intermittently (i.e., in pulses). Pulsing the light device may have advantage over continuous operation because pulsed peak power is higher as compared to cw (average) power and may be useful to break-down the microbe cellular wall. As UV light light interacts with tissue/chromophores to stimulate electron transfer, forming complexes and molecular fragments without generating any heat as contrasted to near and mid IR, pulsing is not required to prevent overheating in the UV range. It is understood that variation in the power of the light source to provide the desired dose to the eye is within the ability of those of skill in the art. For example, dosing can be determined by observing the eye and changes in at least one sign or symptom of the disease or disorder indicating amelioration of the disease or condition. The device can be worn by the subject when standing or sitting. However, it is preferred that the patient is prone during administration of the photoactive agent to the eye. Such considerations are well understood by those of skill in the art.

Representative embodiments of the invention are shown in the figures. Additional features are described in the following description in which embodiments are set forth in detail in conjunction with the accompanying drawings. Numbers on the figures indicate parts in the drawings to which the specification refers.

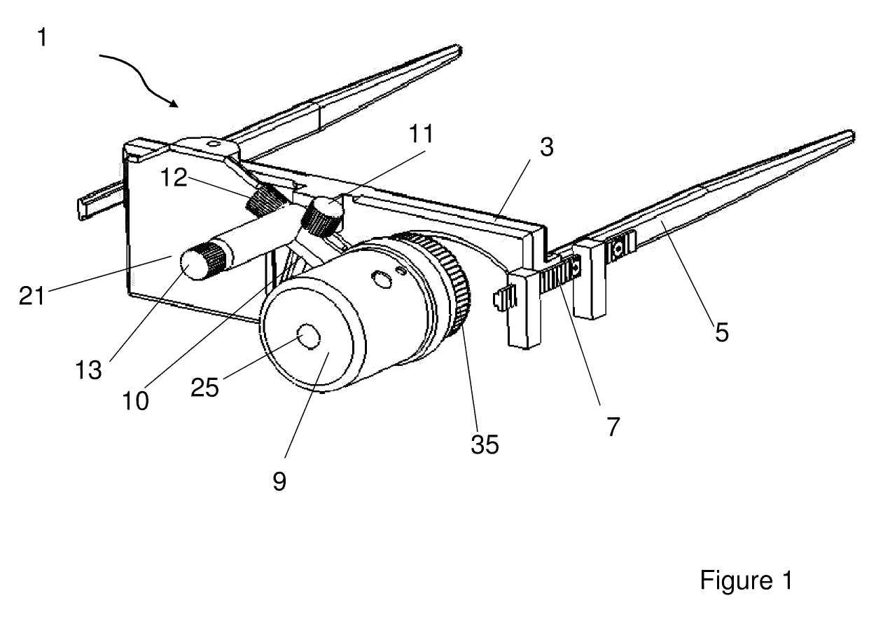

In a preferred embodiment, FIG. 1 is the photoactivator device 1 which includes a frame 3 with a first temple 5 extending from one side of the frame and a second temple 6 extending from the second side of the frame which define a space 8 for receiving the head of a subject and to be held securely in position on the subject. In other embodiments, the device can include a band either alone or in conjunction with temples. The band can extend around, and optionally over the subject's head. In a preferred embodiment, the supports are lightweight and comfortable for use by the wearer. To facilitate adjustment of the device so that the device is held an appropriate distance from the eye, a ruler or other distance gauge 7 can be included in the device, attached to either the frame or the support, or both. The distance gauge can be used for measuring, or to adjust the distance of the frame from the face of the subject. Attached to the frame is a light source contained in a light source housing 9. In a preferred embodiment, the light source housing is attached to the frame using a mount 10 that contains the adjustment knob 11 in conjunction with the adjustment knob 12 to adjust the position of the housing 9 in the x and y direction . Adjustment knob 13 enables the user to adjust the housing of the light source in the z direction to optimize beam location. The mount can includes a set screw on one or more of the adjustment knobs for locking the housing into the desired position. Alternatively, the mount can include ball plungers or other mechanisms to allow for the housing to be retained in a specific position after adjustment. In alternative embodiments, the mount can allow for the position of the housing to be adjusted in one dimension, or two or three dimensions, In certain embodiments, the mount includes markings to allow for the desired position for adjustment of the light source housing for a specific patient to be read and recorded if a patient will undergo multiple treatments with the device. In some embodiments, the light source housing can also be rotated in the mount, or moved back and forth (i.e., closer to or further away from the subject) in the mount. In some embodiments, the light source housing 9 and the occluder 21 can be rotated or swapped to exchange positions so that the light source can treat the right eye. The lens assembly 35 the lenses for focusing the treatment light and can be rotated either clockwise or counterclockwise to select the desired spot size.

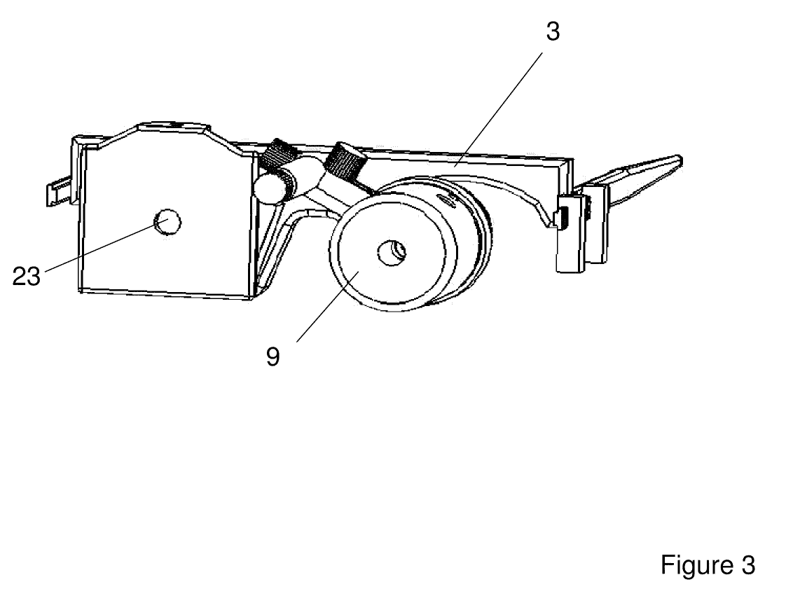

In FIG. 3 a light occluder 21, placed in front of the non-treated eye includes a see-through hole 23 having a diameter of about 6-10 mm to assist the ophthalmologist in registering the light source housing in front of the treatment eye. In another embodiment of this invention the occluder see-through hole 23 allows the patient to view television, a video or other forms of visual entertainment during the treatment. The light source housing can also have a through-hole 25, preferably having a diameter of about 6-10 mm that can hold an applicator head 27 for dispensing a photoactive therapeutic agent, such as riboflavin, to the treatment area.

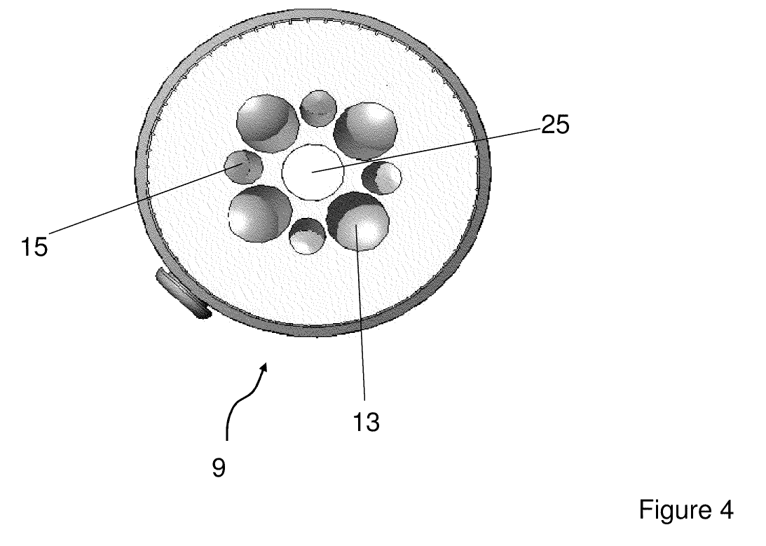

In FIG. 4 the light source housing contains one or more lenses 13 and 15. The lenses may have different sizes (see, e.g., 13 vs 15), but typically have the same focal length 17. In addition, the lenses may be positioned so that the housing is about 1-4 cm, 1.5-2.5 cm, preferably about 2 cm from the cornea. In the embodiment shown, regardless of the size of the lens, each light source is focused to the same spot 19.

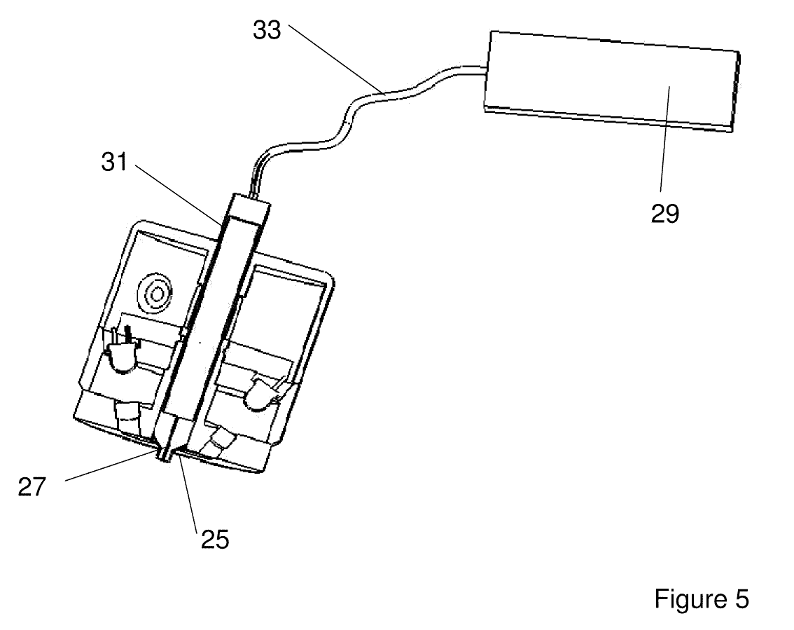

As shown in the embodiment in FIG. 5, application of the photoactive agent to the treatment area can be accomplished by a controller 29, that monitors the number of photosensitizer droplets prior to light exposure, operably connected, for example by tubing 33, to a dripper device 31 attached to the applicator head 27. In an embodiment of the invention, a device (e.g., a blunt needle) for delivery of the photoactive therapeutic agent can be inserted through the opening in the light source housing 25 by a trained individual to deliver the agent to the eye after positioning of the housing, prior to the administration of light.

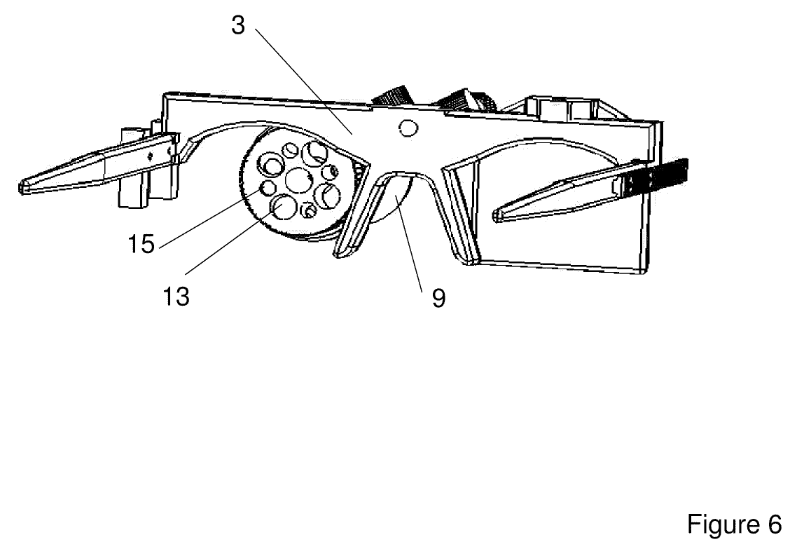

FIG. 6 shows a light source housing with its lenses on the proximal end of the housing mounted on the frame 3.

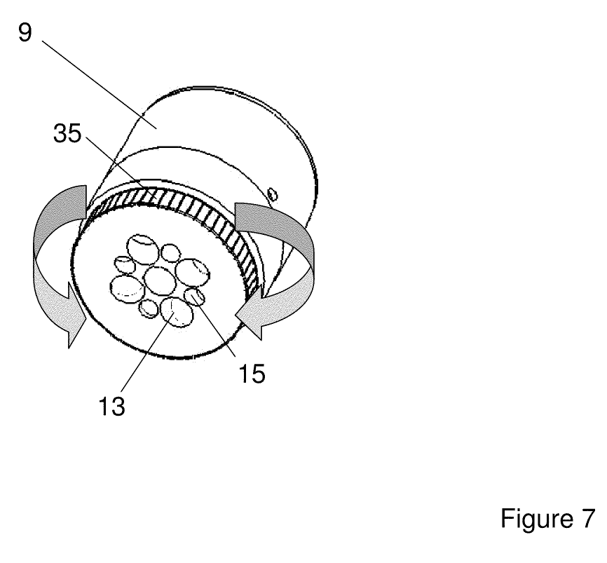

In certain embodiments, such as that shown in FIG. 7 the lens assembly 35 on the proximal end of the light source housing 9 can be rotated as shown by the arrow so that each light source is at the focal point of a lens. Each light source typically aligns with a corresponding lens 13 or 15 during use. Alternatively, the light housing can be used with interchangeable lens assemblies that have different sizes or different numbers of lenses (e.g., 2 or 4) of different sizes. When aligned, each of the light sources will be aligned with a lens of the same size, i.e. the light sources may be aligned with all small diameter or alternatively with all large diameter lenses.

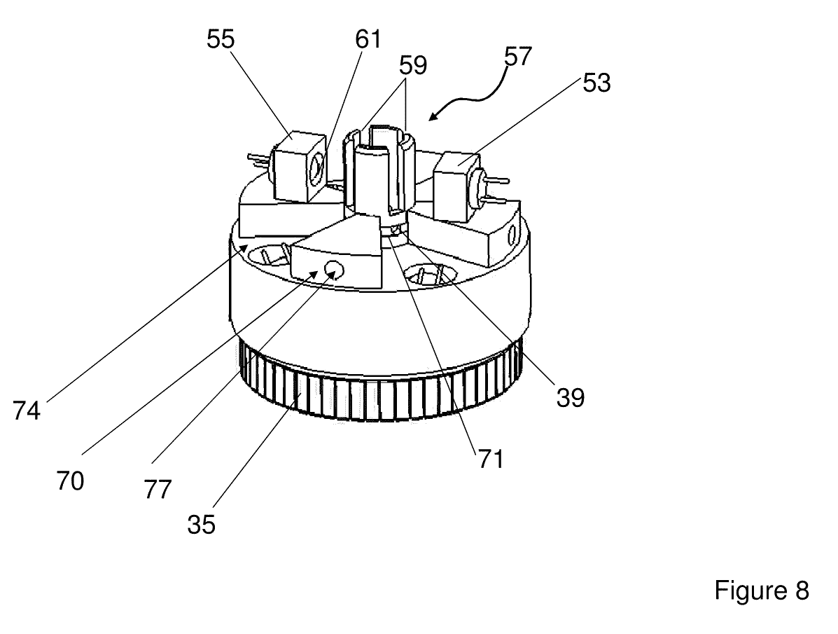

As shown in FIG. 8 and in a preferred embodiment, the lens assembly can be locked into position by a ball plunger assembly 77, actuating into the ball plunger receptacle hole 39 on the light source housing shaft 57. Other locking devices may also be used. On the distal end of the light source housing shaft 57, at least two slots 59 may be provided that align with the positioning holes 61 which allow a NIR source 53-phototransistor 55 pair, for example, to detect which size lens is in position. Transmitted light through the slot is sensed by the phototransistor or another known light sensing device in this embodiment. If light is transmitted through the open slot 59 then the larger diameter lens assembly is selected. If the light is blocked by the shaft 57 then the alternative size lens assembly is in position. As an optional embodiment an integrated NIR source-phototransistor device can be used. In this case if the light path is directed through the slot 59 no light will be detected by the phototransistor and it will remain in an OFF state generating a HIGH signal. In the case that the light is reflected from the shaft 57 then the phototransistor will be in an ON state and will generate a LOW signal. Inspection of FIG. 8 shows the lens assembly 35 is attached to the light source housing shaft 57 such that if the lens assembly is rotated the light source housing shaft 57 also rotates. There are four (4) ball plunger holders 70 positioned 90° from each other located on the top of the holder assembly 74. A set-screw with ball plunger assembly 77 is attached to the ball plunger holder 70. The ball plunger channel 71 is located circumferentially on the light source housing shaft 57 and allows the plunger ball to ride in the channel from ball plunger receptacle hole 39 to the next ball plunger receptacle hole 39. Eight (8) ball plunger receptacle holes 39 are located 45° apart around the ball plunger channel 71. The purpose of the plunger receptacle holes 39 is to lock the selected lens(es) in the lens assembly 35 in place. The phototransistor 55 and the NIR light source 53 are positioned 180° from one another and are centered on a ball plunger holder 70 facing one another.

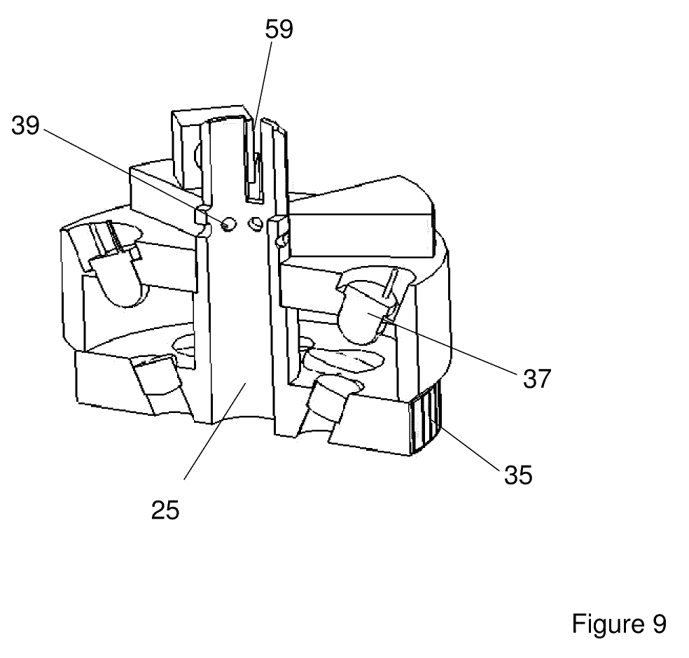

FIG. 9 shows the light source housing in which four individual UVA LED light sources 37 are positioned relative to each of their four 8 mm diameter or their 5 mm diameter lens on the lens assembly 35. The design typically includes positioning the light sources 37 at an angle to one another to optimize beam uniformity within a desired spot size. The light sources 37 are fixed and the lenses may be rotated into position.

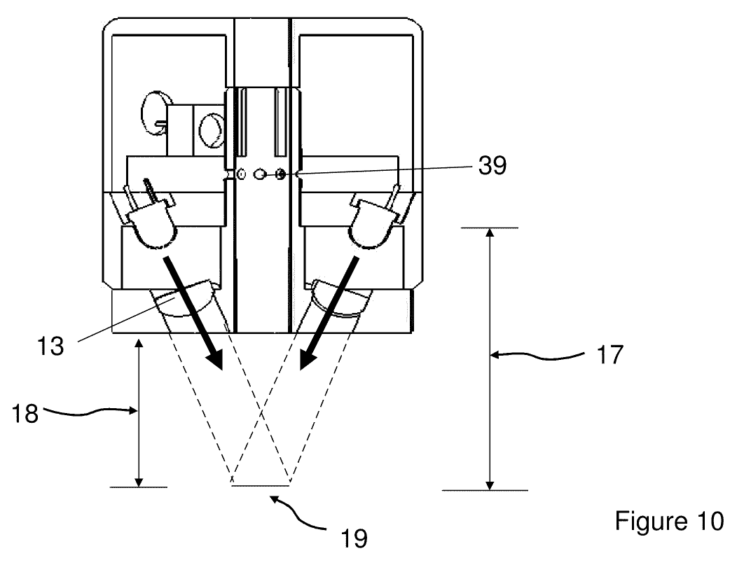

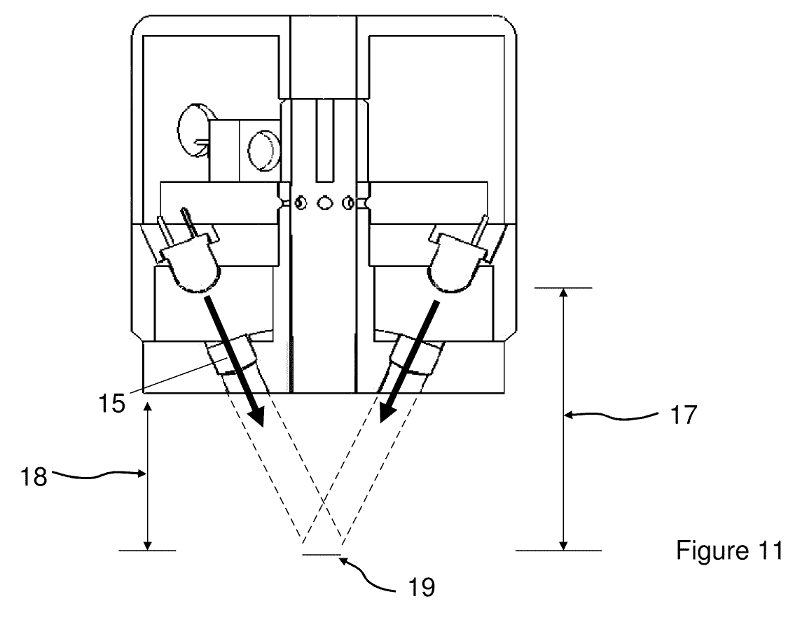

In FIGS. 10 and 11, the lenses produce different spot sizes 19, from 2 to 9 mm using a single light source housing 9 with a rotatable lens housing assembly 35. The working distance 18 is the distance from the distal edge of the light source housing 9 to the subjects eye and can range about 1-4 cm, 1.5-2.5 cm, preferably about 2 cm from the cornea. The focal length 17 is the same for all lenses. In a preferred embodiment of this invention the spot sizes 8 mm in diameter. In embodiments of the invention, the spot size can be about 1, 2, 3, 4, 5, 6, 7, 8, or 9 cm.

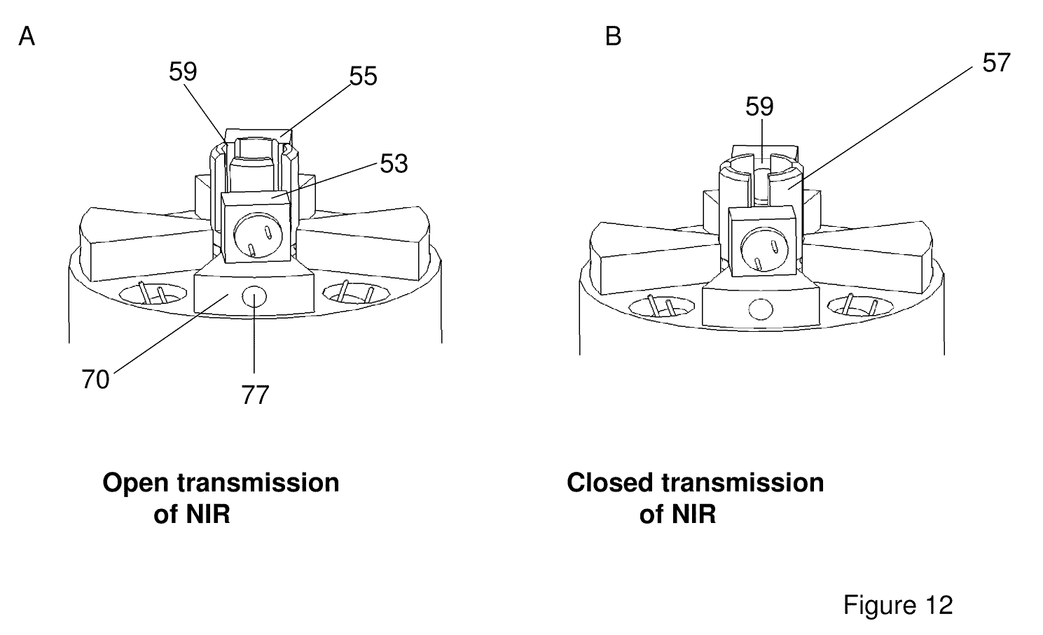

In FIGS. 12A and B a schematic of a power selection mechanism is shown. FIG. 12A is an example of transmission from the NIR source 53 to the phototransistor 55 through slot 59 indicating that the 8 mm diameter lens assembly is selected producing an 8 mm diameter light spot on the eye. In FIG. 12B the NIR source 53 to the phototransistor 55 is blocked by shaft 57 in which case the 5 mm diameter lens assembly is selected thereby producing a 5 mm diameter light spot. The voltage for the NIR source 53 and the voltage for the phototransistor 55 and the signal from the phototransistor 55 are transmitted through cable 51.

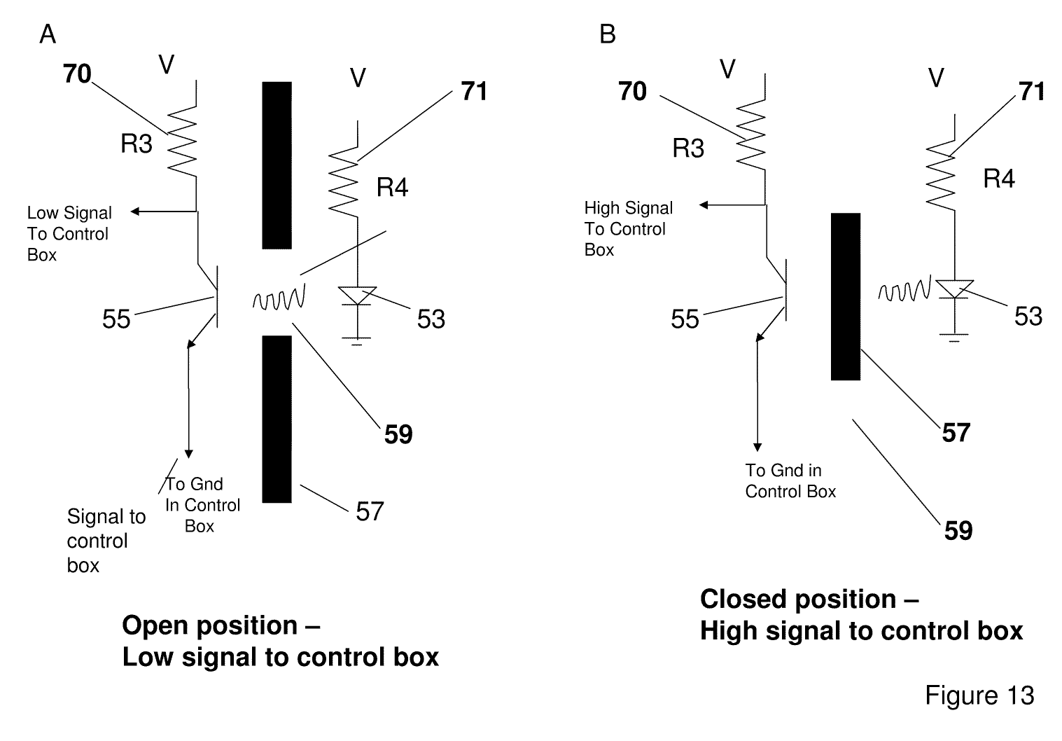

FIGS. 13A and B show a schematic of the control circuit for selection of the lens assembly size. A voltage V is applied to the NIR light source 53. If the slot 59 is open (FIG. 13A) the phototransistor 55 is activated by the NIR light source 53 and a low signal is generated and sent to the control box 41. If the light path from the NIR light source 53 to the phototransistor 55 is blocked by the shaft 57 (FIG. 13B) then a high signal is sent to the control box 41. In FIG. 13 R3 70 is a pull-up resistor and R4 71 is a current limiting resistor.

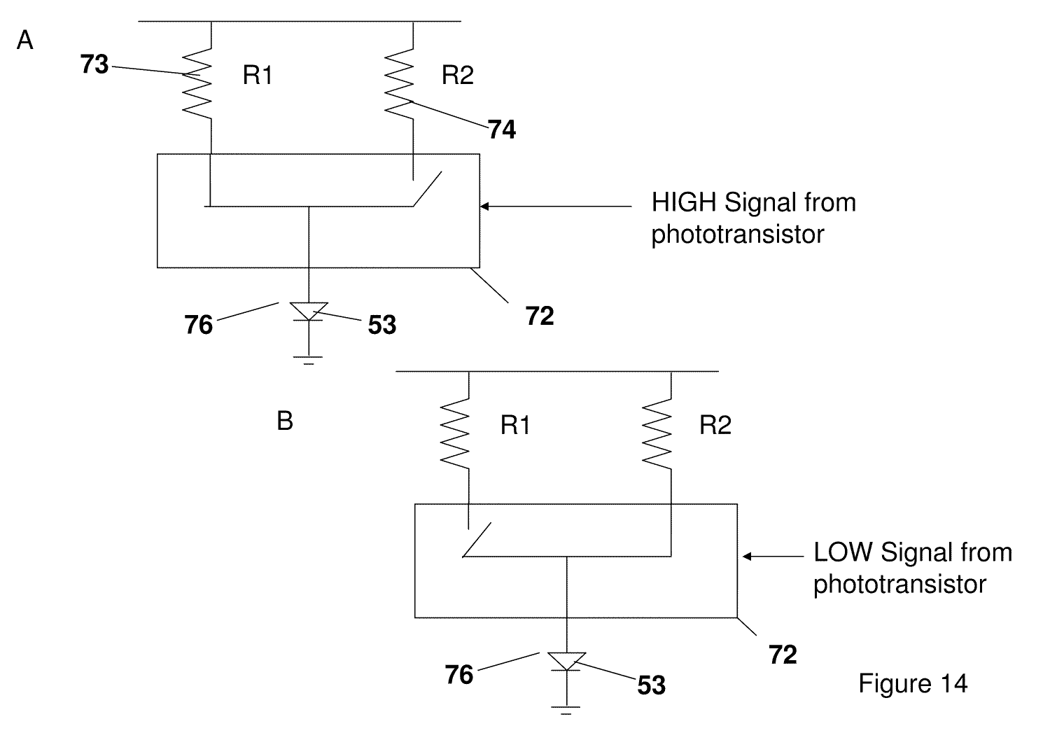

FIGS. 14A and B show schematics for controlling the current through the UVA light sources. A CMOS switch or relay 72 receives the phototransistor signal. A high signal from the phototransistor 55 will select current limiting resistor R1 73 and will control the current through the UVA light sources thereby controlling the power of the UVA light sources through the 5 mm diameter lenses. If the signal from the phototransistor 55 is low then current limiting resistor R2 74 is selected controlling the power of the UVA light sources through the 8 mm diameter lenses. This results in producing the same power density regardless of which size lens assembly is selected. An alternative to the current limiting resistors R1 and R2 is circuitry including operational amplifiers with feedback to produce a constant current source for the UVA light sources.

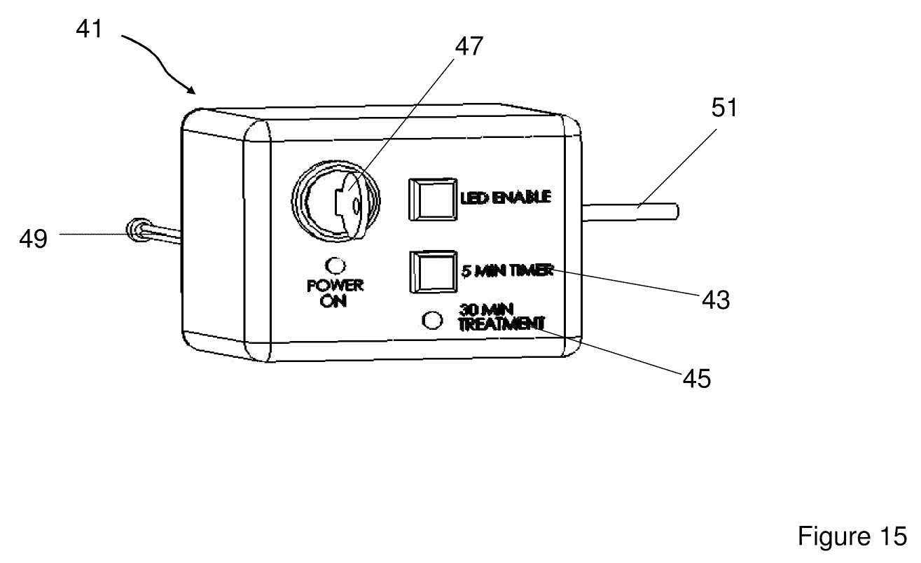

In FIG. 15 the UVA light sources are operationally linked to a controller box 41 via a cable 51. The power output optionally includes one or more timer devices 43 and 45, a key or other power switch 47, which applies 120V AC power to the controller box via power cable 49 with an indicator light 40 showing that the power is ON. Within the controller box 41 is a power supply that will supply voltage and current to UVA light sources and the NIR light source 53 and phototransistor 55 and circuitry to detect signals from the phototransistor 55. The controller box 41 can include a microprocessor or logic arrays, displays and selectable input functions. In an alternative embodiment to this invention each light source may have its own control circuit to maintain a stable, constant drive current for a constant output power. In addition, each light source may have the output signal from its rear facet photodetector that can be sensed by control circuitry contained in the control box and used in a feedback circuit to maintain a constant power output from each respective light source. The control box can include interlocks to prevent inadvertent powering of the unit.

The light source drive currents are typically selected to produce the desired power density for each selected spot size. Known in the art, each light source may have a rear facet photodetector or other light sensing device. The photodetector output signal can provide feedback to maintain a constant power output. The light source can have a constant current drive source to maintain a constant and stable power output.

### Kits

The invention further provides kits for practicing the methods of the invention. Kits of the invention include, for example, a device of the invention and a photoactive therapeutic agent. Kits can further include instructions for use and appropriate packaging. Kits of the invention can include replacement bulbs, interchangeable lens housings, various size supports to allow the device to be adjustable, and other components for practicing the methods of the invention.

The following examples are put forth so as to provide those of ordinary skill in the art with a complete disclosure and description of how to make and use the assay, screening, and therapeutic methods of the invention, and are not intended to limit the scope of what the inventors regard as their invention.

This invention is further illustrated by the following examples, which should not be construed as limiting.

### EXAMPLES

### Example 1

### Treatment of Corneal Infection Using UV-A Photoactive Therapeutic Agent and a Wearable Photoactivator

A patient presents with acanthamoebic keratitis in one eye. A lid speculum is used to hold the patient's eye open. The recumbent patient is fitted with a wearable photoactivator of the invention having a UV-A light source. The housing of the light source is adjusted to provide light over 3 to 10 mm spot size on the eye, depending on the area to be exposed, based on the extent of the infection. The fluence of the light is such that it warrants its absorption in the layers of the cornea before penetrating into other ocular structures, thereby reducing the exposure of other structures to the light. A dropper is inserted through an opening in the housing to apply riboflavin to the eye in the form of drops and the riboflavin solution concentration is in the range of about 0.1% to 5% to completely bathe the eye in riboflavin.

The riboflavin is instilled in the eye every 5 minutes for 15 minutes in the form of eyedrops or soaked in a filter paper disc placed on the surface of the cornea in order to impregnate the stroma with the photochemical substance. The light source is then turned on for a period of 30 minutes, with continuous instillation of riboflavin eyedrops every 5 minutes. The fluence used is in the range of 3 to 20 mW/cm². The spot of UV-A light is adjusted according to the size of the ulcer, and centered in the area of infection to cover the entirety of the corneal infiltrate caused by the keratitis. The limbal area can be protected by a mask to avoid exposure of this area and prevent further damage in the limbal stem cell pool.

### Example 2

### Treatment of Keratoconus Using UV-A Photoactive Therapeutic Agent and a Wearable Photoactivator

A patient presents with keratoconus in one eye. A lid speculum is used to hold the patient's eye open. The recumbent patient is fitted with a wearable photoactivator of the invention having a UV-A light source. The optical system for the light source is adjusted to provide light of 8-9 mm spot size on the eye. The fluence of the light is such that it warrants its absorption in the layers of the cornea before penetrating into other ocular structures. A dropper is inserted through an opening in the housing to apply riboflavin to the eye in the form of drops and the riboflavin solution concentration is in the range of 0.1% to 5% to completely bathe the eye in riboflavin.

The riboflavin is instilled in the eye every 5 minutes for 15 minutes in the form of eyedrops or soaked in a filter paper disc placed on the surface of the cornea to impregnate the sroma with the photochemical substance. The light source is then turned on for a period of 30 minutes, with continuous instillation of riboflavin eyedrops every 5 minutes. The fluence used is in the range of 3 to 20 mW/cm², and the riboflavin solution concentration is in the range of 0.1% to 5%. The spot of UV-A light will be adjusted according to the size of the ulcer, and centered in the cornea to cover the majority of the corneal area. The limbal area may be protected by a mask to avoid exposure of this area and prevent further damage in the limbal stem cell pool.

What is claimed is:

1. A wearable ocular photoactivator device comprising:
a frame to position on a face of a subject a light source towards an eye of the subject; wherein
the light source enclosed in a housing is connected to the frame wherein the housing comprises
the light source contained within the housing, and
a lens capable of alignment with emitted light from the light source to direct the emitted light to the eye of the subject; and
a power source operably connected to the light source.

2. The device of claim 1, wherein the frame further comprises a structure for attachment of the frame a head of the subject.

3. The device of claim 2, wherein the structure for attachment of the frame to the head of the subject is selected from the group consisting of arms and bands.

4. The device of claim 1, wherein the housing is connected to the frame using a mount.

5. The device of claim 4, wherein the mount is adjustable.

6. The device of claim 1, wherein the light source is selected from the group consisting of light emitting diode (LED), laser diode, frequency tripled Nd:Yag solid state laser, dye laser, quartz lamp, fluorescent lamp, Nernst glower, Tungsten-Halogen lamp, and discharge lamp.

7. The device of claim 1, wherein the light emitted is selected from the group consisting of ultraviolet, visible, and infrared.

8. The device of claim 7, wherein the wavelength of light emitted is UV-A (380 nm -315 nm).

9. The device of claim 1, wherein the housing comprises more than one lens.

10. The device of claim 9, wherein the lenses comprise different size lenses.

11. The device of claim 10, wherein a portion of the housing is rotatable to align at least one lens with the light source.

12. The device of claim 1, wherein the housing further comprises an opening through the housing to allow observation of an eye of the subject, when the subject is wearing the device.

13. The device of claim 12, further comprising a delivery device for insertion through the opening in the housing.

14. The device of claim 1, wherein the device further comprises a distance gauge that measures a distance from the light source housing to a portion of the eye of the subject.

15. The device of claim 1, wherein the device further includes an occluder.

16. The device of claim 15, wherein the occluder comprises an opening.

17. The device of claim 1, wherein the device further comprises an integrated phototransistor and NIR source to detect light emitted by the light source.

18. The device of claim 1, wherein the lens angle is adjustable in the housing.

19. A method of exposing an eye of a subject to light, the method comprising:
providing the device of claim 1,
positioning the housing to direct light to the eye of the subject, and
providing power to the device to emit light to the eye of the subject, whereby the eye of the subject is exposed to light.

20. The method of claim 19, wherein the method comprises treatment of a disease or disorder.

21. The method of claim 20, wherein the disease or disorder is selected from the group consisting of ocular infection, corneal infection, keratoconus, corneal ectasias, corneal dystrophy, and corneal neoplasia.

22. The method of claim 19, further comprising administration of a photoactive therapeutic agent to the subject prior to exposure to the light.

23. The method of claim 22, wherein the agent is administered topically to the eye.

24. The method of claim 22, wherein the photoactive therapeutic agent is selected from the group consisting of riboflavin, psoralen, lumiflavin, lumichrome, rose Bengal, eosin, courmarin, sparfloxacin, fluorescein, ficusin, psoberan, Toluidine Blue O, methylene blue, and thionin.

25. A kit comprising a device of claim 1 and a photoactive therapeutic agent.
